(19) United States
(12) Patent Application Publication
(10) Pub. No.: US 2025/0269370 A1
(43) Pub. Date: Aug. 28, 2025

(54) MICROFLUIDIC URINE ALBUMIN/CREATININE CHIP (uACR-CHIP) FOR CHRONIC KIDNEY DISEASE EVALUATION

(71) Applicant: University of Manitoba, Winnipeg (CA)

(72) Inventors: Francis Lin, Winnipeg (CA); Dumitru Tomsa, Winnipeg (CA); Amanda Stefanson, Winnipeg (CA); Xiaoou Ren, Winnipeg (CA); Yang Liu, Winnipeg (CA); Claudio Rigatto, Winnipeg (CA); Paul Komenda, Winnipeg (CA); Navdeep Tangri, Winnipeg (CA)

(21) Appl. No.: 18/857,751

(22) PCT Filed: Apr. 17, 2023
(86) PCT No.: PCT/CA2023/050517

§ 371 Date: Oct. 17, 2024

Related U.S. Application Data
(60) Provisional application No. 63/333,054, filed on Apr. 20, 2022.

(51) Int. Cl. B01L 3/00; G01N 21/64; G01N 33/68; G01N 33/70

(52) U.S. Cl. CPC B01L 3/502715; B01L 3/502738; G01N 21/6428; G01N 33/6893; G01N 33/70; B01L 2200/0684; B01L 2200/16; B01L 2300/047; B01L 2300/0867; B01L 2300/0883; G01N 2021/6439; G01N 2333/765; G01N 2800/347

(57) ABSTRACT

Provided is a microfluidic device for measurement of albumin and creatinine in a biological sample, such as urine, and methods for fabricating such devices. Also provided are methods of detecting albumin and creatinine and a ratio of albumin to creatinine in a biological fluid sample to diagnose or monitor a chronic kidney disease.

### RELATED APPLICATION

Benefit of priority is claimed to U.S. Provisional Application No. 63/333,054, titled “MICROFLUIDIC URINE ALBUMIN/CREATININE CHIP (uACR-CHIP) FOR CHRONIC KIDNEY DISEASE EVALUATION,” filed Apr. 20, 2022.

Where permitted, the subject matter of the above-referenced application is incorporated by reference in its entirety.

### TECHNICAL FIELD

The present invention relates to microfluidic devices, and methods and systems using such devices. Provided are devices that manipulate, process, or otherwise alter micro-sized amounts of fluids and fluid samples; microfluidic analysis equipment; and to a point of care diagnostic device, for measurement of albumin and/or creatinine in urine.

### BACKGROUND

CKD is a global health epidemic, afflicting more than 850 million people world-wide, and is a potent risk factor for kidney failure, heart disease and death. The cause of CKD varies but some of the most common factors include diabetes, high blood pressure and cardiovascular disease. There are few signs or symptoms in the early stage of CKD, which makes early diagnosis difficult. CKD can progress to end-stage kidney failure if it is not treated properly, a condition that is burdensome and expensive to treat, and potentially fatal. Fortunately, effective treatments exist which can prevent or delay catastrophic downstream outcomes if CKD is diagnosed early. Optimal detection and risk assessment of CKD requires simultaneous estimation of both kidney function (e.g., glomerular filtration rate [GFR]) and kidney damage (e.g., albuminuria).

Albuminuria is a pathological condition wherein the protein albumin is abnormally present in the urine. The gold standard method for measuring albuminuria excretion in the urine is the timed 24-hour urine collection. In clinical settings, this is awkward and burdensome for patients, and prone to errors of both under and over collection unless performed in a research setting. Measurement of both albumin and creatinine concentration simultaneously in a single sample of urine and calculation of the albumin to creatinine ratio (ACR), expressed as mg/mmol, has replaced the measurement of 24-hour urine albumin excretion clinically, and is recommended for use in screening and diagnosis of kidney disease in multiple guidelines. An ACR of <2.8 mg/mmol is normal, a ratio of 2.8-28 mg/mmol represents microalbuminuria, and a ratio >28 mg/mmol represents macroalbuminuria. The ACR is tightly correlated with the 24-hour urine result and is highly predictive of outcomes in multiple clinical settings, including, but not limited to, kidney disease, heart failure, cancer, and diabetes. Routine diagnosis and staging of chronic kidney disease (CKD) therefore requires an evaluation of the albumin to creatinine ratio in patient urine in addition to blood chemistry analysis for estimation of GFR.

The advantage of the urine ACR is that it can be performed in a single urine sample as opposed to timed collection over 24 hours. Moreover, urine collection is non-invasive, which makes it an ideal sample for point-of-care (POC) detection. The disadvantage is that it requires quantitation of both albumin and creatinine in the urine, as opposed to albumin alone.

The current standard for urine creatinine quantification uses a bench top chemistry analyzer which is large and expensive. A number of different chemistries for the detection of creatinine can be used, including the Jaffe reaction described below.

Albumin can be measured immunologically or non-immunologically. Immunologically-based laboratory methods include immunonephelometry, immunoturbidimetry, and radioimmunoassay. Conventional, non-immunologic, fluorescence-based assays for detecting albumin are also available and require mixing a reacting dye with the test sample at a precise, fixed ratio in a well-plate, waiting for several minutes for the mixture to react, and measuring the fluorescent intensity from the reaction product to evaluate the albumin concentration. Such a method requires precise solution metering equipment such as a pipette in order to deliver the precise mixing ratio. In addition, the time window for detection is short, usually requiring reading of the fluorescence signal within 5 minutes of the reaction starting, because after 5 minutes, the fluorescence signal changes due to overreaction and evaporation, which negatively impacts the accuracy of the measurement. Furthermore, the well-plate method requires relatively large volumes of reagents (for example, tens of microliters per well), trained personnel, and typically a designated laboratory in which to perform the assays.

While inexpensive point-of-care methods (e.g., dipstick tests) do exist and are widely available for detecting albumin in a urine sample, the limit of detection (LOD) and accuracy of these dipstick tests is usually not good, and therefore they are not sufficiently quantitative and lack adequate precision. At present, inexpensive point of care tests for urine creatinine do not exist. A POC device and method for the detection and measurement of creatinine and/or albumin levels in a urine sample of a subject that optimally balances accuracy, cost, simplicity and low facility requirement, would be a highly desirable tool to effectively address the epidemic of CKD worldwide.

Accordingly, a need exists for methodologies and devices that allow for the detection and measurement of creatinine and/or albumin levels in a urine sample of a subject that addresses the disadvantages of existing technologies.

### SUMMARY

Provided herein is a low cost, microfluidic Lab-on-Chip platform for the simultaneous measurement of creatinine and albumin concentrations in microliter volumes of the same urine sample.

In one embodiment, provided is a pump-free, passive-flow PDMS-based microfluidic device designed to draw a single small volume urine sample (microliter scale sample) into two parallel and separate bioassays that employ completely different reagents for the separate quantification of target biomolecules (albumin and creatinine). Rather than requiring precise volume control typically employed in bioassays, the precision of the uACR-Chip provided herein employs ratiometric mixing of the urine sample with the reaction reagents at defined ratios for defined reaction times in two separate but integrated microfluidic assay designs. Volumes, flows, and times are determined by the unique design of the microfluidic channels. Distinct observation windows into the microfluidic reaction chambers allow separate optical measurements (e.g., absorbance or fluorescence) of a time-dependent reaction at defined time points to determine the creatinine and albumin concentrations in the sample. The observation windows have been designed for rapid and uniform filling, and air release valves have been engineered to facilitate fluid filling of the empty chip, eliminating bubble formation. The albumin: creatinine ratio (ACR) can be calculated from the albumin and creatinine measurements.

The uACR-Chip provided herein solves the problems associated with conventional testing techniques because of its 1) low cost, 2) quantitative and stable measurement, 3) easy operation, and 4) suitability for use with inexpensive and portable optical readers. These attributes are unique compared to existing methods. The uACR-Chip can be integrated with a reading device to provide POC detection of albumin and creatinine and quantification of the albumin: creatinine ratio as a disease biomarker for chronic kidney disease. The uACR-Chip can be used in a lab-on-a-chip point-of-care device. Such devices can allow medical analyses to be conducted at or near the site of the patient care.

According to another aspect of the invention, provided is a method for detecting albumin and creatinine in a urine sample to diagnose or monitor a chronic kidney disease, the method including using the uACR chip provided herein to detect and quantify the amount of albumin and creatinine in a urine sample.

According to another aspect of the invention, provided is a microfluidic device, comprising (a) a first reagent flow path that includes a first reagent inlet well; a first reagent channel having a depth, a width and a length, in fluid communication with the first reagent inlet well, the first reagent channel having a first reagent flow rate; a first mixing chamber in fluid communication with the first reagent channel; a first reaction channel in fluid communication with the first mixing chamber; a first reaction detection window in fluid communication with the first reaction channel; a first reagent outlet well in fluid communication with the first reaction detection window via a first outlet channel; and at least one air release valve; (b) a second reagent flow path that includes a second reagent inlet well; a second reagent channel having a depth, a width and a length, in fluid communication with the second reagent inlet well, the second reagent channel having a second reagent flow rate; a second mixing chamber in fluid communication with the second reagent channel; a second reaction channel in fluid communication with the second mixing chamber; a second reaction detection window in fluid communication with the second reaction channel; a second reagent outlet well in fluid communication with the second reaction detection window via a second outlet channel; and at least one air release valve; (c) a sample inlet well; (d) a first sample flow path that includes a first sample channel having a depth, a width and a length, in fluid communication with the sample inlet well and the first mixing chamber of the first reagent flow path, the first sample flow path having a first sample flow path flow rate; (e) a second sample flow path that includes a second sample channel having a depth, a width and a length, in fluid communication with the sample inlet well and the second mixing chamber of the second reagent flow path, the second sample flow path having a second sample flow path flow rate; and (f) an oil confiner having a depth, width, and length encompassing the first reagent inlet well, the second reagent inlet well, and the sample inlet well to form a trough for containing an oil in fluid communication with each of the first reagent inlet well, the second reagent inlet well, and the sample inlet well.

In the microfluidic device provided herein, the first reaction detection window and the second reaction detection window each separately can have a shape that is elliptic, biconvex, lenticular, fusiform, ovate, lanceolate, oblanceolate, or tear-drop. The first reaction detection window and the second reaction detection window each can include a window flow diverter. The window flow diverter in the first reaction detection window can have a shape that is elliptic, biconvex, lenticular, fusiform, ovate, lanceolate, oblanceolate, or tear-drop; and the window flow diverter in the second reaction detection window can have a shape that is elliptic, biconvex, lenticular, fusiform, ovate, lanceolate, oblanceolate, or tear-drop. In the microfluid devices provided herein, the first reaction detection window can have a width of about 150 to 3,000 μm and a length of about 150 to 6,000 μm; and the second reaction detection window can have a width of 150 to 3,000 μm and a length of about 150 to 6,000 μm.

In the microfluid devices provided herein, a width of the first reagent channel can be from about 50 to 150 μm; a width of the second reagent channel can be from about 50 to 150 μm; a width of the first sample channel can be from about 50 to 150 μm; and a width of the second sample channel can be from about 50 to 150 μm. A depth of the first reagent channel can be from about 50 to 150 μm; a depth of the second reagent channel is from about 50 to 150 μm; a depth of the first sample channel is from about 50 to 150 μm; and a depth of the second sample channel is from about 50 to 150 μm.

In the microfluid devices provided herein, the first mixing chamber is tapered and can have a width at it widest point of 200 to 500 μm and a width at its narrowest point of 100 to 150 μm; and the second mixing chamber is tapered and can have a width at it widest point of 200 to 500 μm and a width at its narrowest point of 100 to 150 μm. The first reagent channel and the first sample channel are connected to the first mixing chamber at opposite positions so that a fluid stream from the first reagent channel and a fluid stream from first sample channel converge within the first mixing chamber to create turbulence; and the second reagent channel and the second sample channel are connected to the second mixing chamber at opposite positions so that a fluid stream from the second reagent channel and a fluid stream from the second sample channel converge within the second mixing chamber to create turbulence.

In the microfluid device provided herein, the first reagent channel and the second reagent channel along the major part of their length can have the same cross-sectional shape. The length of the first reagent channel and the length of the second reagent channel each independently can be about 5 to 150 mm. The first reagent channel and the second reagent channel each independently can include at least one section having a zig zag shape comprising turns at right angles. The depth of the first reaction channel can be from about 50 to 1000 μm; and the depth of the second reaction channel is from about 50 to 1000 μm. The width of the first reaction channel can be from about 50 to 1000 μm; and the width of the second reaction channel can be from about 50 to 1000 μm. The length of the first reaction channel can be from about 5 to 150 mm; and the length of the second reaction channel is from about 5 to 150 mm. A width of the first outlet channel can be about 110% to 500% of the width of the first reaction channel; and a width of the second outlet channel is about 110% to 500% of the width of the second reaction channel.

The microfluid device provided herein can include at least one air release valve. In some embodiments, each flow path can include one or more than one air release valve. In some embodiments, at least one air release valve can be located before and in close proximity to the first reaction detection window, and at least one air release valve can be located before and in close proximity to the second reaction detection window. In some embodiments, an air release valve can be located after and in close proximity to the first mixing chamber, and an air release valve can be located after and in close proximity to the second mixing chamber. In some embodiments, the microfluidic device provided herein can include a first reagent flow path that can include a first air release valve that can be located before and in close proximity to the first reaction detection window, and a second air release valve that can be located after and in close proximity to the first mixing chamber. In some embodiments, the microfluidic device provided herein can include a second reagent flow path that can include a first air release valve that can be located before and in close proximity to the second reaction detection window, and a second air release valve that can be located after and in close proximity to the second mixing chamber.

Also provided herein are methods of detecting albumin and creatinine in a single drop of biological fluid sample to diagnose or monitor a chronic kidney disease, the method including providing a microfluid device as provided herein; applying a solution of a fluorescent dye for detection of albumin to the first reagent inlet well; applying a biological sample to the sample inlet well; applying a solution of a dye for detecting creatinine to the second reagent inlet well; applying an oil to the oil confiner to completely cover the reagent inlet wells and the sample inlet well; allowing the solution of a fluorescent dye for detection of albumin and the biological sample to mix in the first mixing chamber to form a first reaction product that flows to the first detection window; allowing the solution of the dye for detection of creatinine and the biological sample to mix in the second mixing chamber to form a second reaction product that flows to the second detection window; detecting a detectable signal in the first detection window; and detecting a detectable signal in the second detection window. In the methods, the oil has a density lower than a density of the solution of a fluorescent dye for detection of albumin, a density of the solution of the dye for detection of creatinine, and the biological sample so that the oil floats on top of a surface of the solution of a fluorescent dye for detection of albumin, the solution of the dye for detection of creatinine, and the biological sample. In the methods, the biological sample can be or is urine.

In the methods of detecting albumin and creatinine provided herein, a fluorescent dye can be used for the detection of albumin, and a fluorescent dye or colorimetric dye can be used for the detection of creatinine. In some methods, the fluorescent dye for detection of albumin can include albumin blue 580 Potassium salt, Square-655 dye, Square-680-Carboxy dye, Nile Red, 8-anilino-1-naphthalenesulfonic acid (ANS), FITC-dextran, rhodamine, bromocresol green (BCG), bromocresol purple (BCP), Texas Red, or a combination thereof. In some methods, the dye for detection of creatinine can include picric acid.

Also provided are kits that include a microfluidic device as provided herein, and instructions for the use thereof. The kit can also include a solution of a fluorescent dye for detection of albumin; and a solution of a dye for detection of creatinine. The kit can include a fluorescent dye for detection of albumin that includes albumin blue 580 Potassium salt, Square-655 dye, Square-680-Carboxy dye, Nile Red, 8-anilino-1-naphthalenesulfonic acid (ANS), FITC-dextran, rhodamine, bromocresol green (BCG), bromocresol purple (BCP), Texas Red, or a combination thereof. The kit can include a dye for detection of creatinine that includes picric acid.

### DESCRIPTION OF THE DRAWINGS

The accompanying figures are included to provide a further understanding of the present invention, and are incorporated in and constitute a part of this specification. The drawings illustrate exemplary embodiments of the present invention and, together with the description, serve to explain principles of the present invention.

The drawings are not to scale and may not exactly represent the actual layout or exact shape of a particular element in the design.

FIGS. 1A and 1B are schematic plan views of exemplary embodiments of the microfluidic device of the present invention.

FIG. 2 is a schematic plan view of exemplary embodiment of a mixing chamber of the microfluidic device of the present invention.

FIG. 3A is a schematic plan view of a configuration of a conventional detection window having a diamond shape.

FIG. 3B is a schematic plan view of a configuration of a conventional detection window having a square shape.

FIG. 4 is a schematic plan view of several exemplary configurations of detection windows as described herein having a smooth curved configuration.

FIG. 5 is a schematic plan view of several exemplary configurations of detection windows containing one or more than one flow diverter as described herein.

FIG. 6A is a photograph showing the filling of a conventional simple square shape detection window, and FIG. 6B is a photograph showing the filling of a lenticular shaped detection window containing a flow diverter as described herein.

FIG. 7 shows the velocity profile for filling of a conventional simple square shape detection window.

FIG. 8 shows the velocity profile for filling of a lenticular shape detection window that includes a flow diverter as described herein.

FIG. 9 shows a schematic drawing of a detection window that does not include a flow diverter, and the corresponding filling velocity profile for the detection window.

FIG. 10 shows a schematic drawing of a detection window that includes a flow diverter as described herein, and the corresponding filling velocity profile for the detection window.

FIG. 11 is a photograph plan view of exemplary embodiment of the microfluidic device of the present invention in use.

FIG. 12 is a graph showing calibration of the albumin detection part of an exemplary uACR-Chip provided herein, demonstrating a limit of detection (LOD) of 4.54 μg/mL.

FIG. 13 is a graph showing calibration of the creatinine detection part of an exemplary uACR-Chip provided herein, demonstrating a limit of detection (LOD) of 0.41 mM.

FIG. 14 is a graph showing signal stability over time for the albumin/dye reaction in the uACR-Chip provided herein for different concentrations of albumin.

FIG. 15 is a graph showing signal stability over time for the creatinine/dye reaction in the uACR-Chip provided herein for different concentrations of creatinine.

### DETAILED DESCRIPTION

### Definitions

Unless defined otherwise, all technical and scientific terms used herein have the same meaning as is commonly understood by one of skill in the art to which the inventions belong. All patents, patent applications, published applications and publications, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the singular forms “a,” “an” and “the” include plural referents unless the context clearly dictates otherwise.

As used herein, all ranges include the upper and lower limits. As used herein, the recitation of a numerical range for a variable is intended to convey that the variable can be equal to any value(s) within that range, as well as any and all sub-ranges encompassed by the broader range. Thus, the variable can be equal to any integer value or values within the numerical range, including the end-points of the range. As an example, a variable which is described as having values between 0 and 10, can be 0, 4, 2-6, 2.75, 3.3-4.4, etc.

As used herein, “about” is a term of approximation and is intended to include minor variations in the literally stated amounts, as would be understood by those skilled in the art. Such variations include, for example, standard deviations associated with techniques commonly used to measure the amounts of the constituent elements or components of an alloy or composite material, or other properties and characteristics. All of the values characterized by the above-described modifier “about,” are also intended to include the exact numerical values associated therewith. Hence “about 5 percent” means “about 5 percent” and also “5 percent.”

As used herein, “optional” or “optionally” means that the subsequently described event or circumstance does or does not occur, and that the description includes instances where said event or circumstance occurs and instances where it does not. For example, an optional component in a system means that the component may be present or may not be present in the system.

As used herein, the terms “comprises” and “comprising” are inclusive and open ended, and not exclusive. When used in the specification and claims, the terms “comprises” and “comprising” and variations thereof mean the specified features, steps or components are included, but do not exclude other features, steps or components.

Any compositions described herein are intended to encompass compositions which consist of, consist essentially of, as well as comprise, the various constituents identified herein, unless explicitly indicated to the contrary.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

As used herein, the term “exemplary” means “serving as an example or illustration,” and should not be construed as being preferred or advantageous over other configurations disclosed herein.

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

As used herein, the term “subject” includes members of the animal kingdom including but not limited to human beings.

As used herein, the fluid introduced into a microfluidic device for testing can be referred to a “sample fluid.”

As used herein, “analyte” means a substance of interest whose presence is being identified or whose concentration is being measured.

As used herein, a “microfluidic device” refers to a system, device, or device component that contains a liquid pathway configured to hold a fluid and to have at least one physical dimension of 1000 microns or less.

As used herein, “room temperature” refers to a temperature of about 20° C.

As used herein, “close proximity to X” refers to being at a location that is about 5,000 microns or less from X.

### Multi-Pathway Microfluidic Device

Microfluidics has been applied to a wide range of applications, but device design remains a significant challenge. The microfluid devices provided herein can use techniques similar to those used to form printable circuit boards, or lithographic methods, for their production, using known techniques and materials. For example, a stamp or a wafer die containing a desired pattern of a microfluid device, can be used to produce the desired features of the microfluid device.

In one example, provided is a microfluidic urinary creatinine/albumin chip (uACR-Chip) that exploits the nonimmunological assays for determining the amount of albumin and creatinine in a single urine sample. The uACR-Chip provided herein is a passive and continuous mixing module, in which the loading process requires only an inexpensive dropper, no external equipment is required to apply a pressure to the microfluidic device, and the resulting signal generated by the reactions in the flow paths of the device are stable over time, as discussed in more detail below. The uACR-Chip provided herein utilizes a pressure-balancing strategy based on an immiscible oil that covers the reactant and sample wells, which highly improves the precision in controlling the mixing ratio of sample and dye, resulting in more robust signal development.

Preferred embodiments of the present invention will be described below in more detail with reference to the accompanying drawings. The present invention can, however, be embodied in different forms and should not be construed as limited to the exemplified embodiments set forth herein.

### Exemplary Device

Provided is a microfluid device for precise measurement of albumin and creatinine concentration in a drop of urine (uACR-Chip), which can be used to calculate the ratio of albumin to creatinine (ACR) in the sample.

In the uACR-Chip provided herein, a single chip includes different reagent/reaction channel combinations. One set of the reaction channels is arranged to carry out a specific detectable reaction, while a second set of reaction channels is arranged to carry out a second detectable reaction. For example, a single urine sample of a subject can be simultaneously subjected to two different tests, one for the measurement of albumin levels, and one for the measurement of creatinine levels.

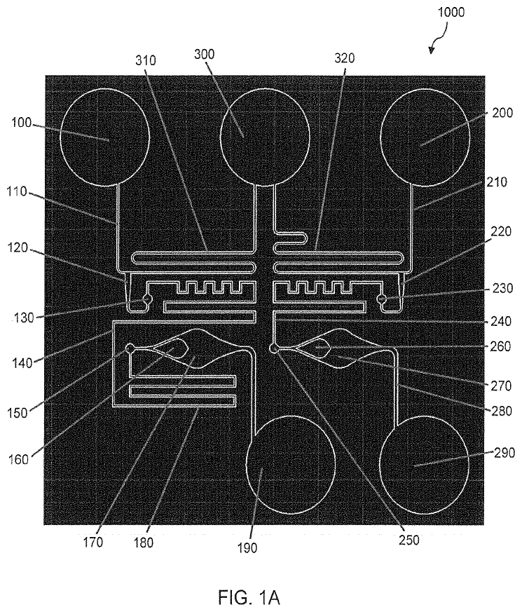

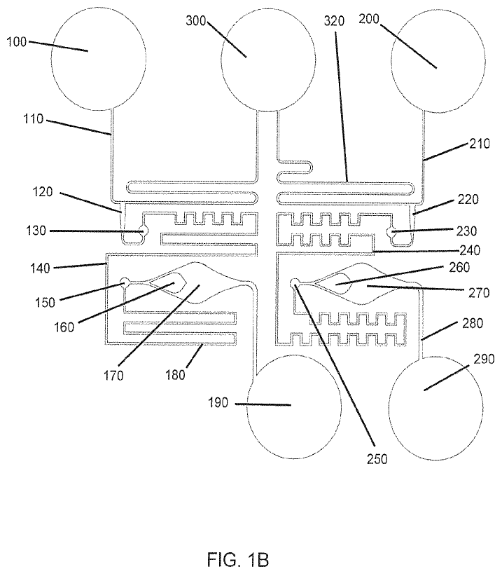

An exemplary embodiment of the uACR-Chip provided herein is shown in FIG. 1. In the exemplary embodiment shown, the uACR-Chip can include:
(a) a first reagent flow path comprising:
   a first reagent inlet well;
   a first reagent channel having a depth, a width and a length, in fluid communication with the first reagent inlet well, the first reagent channel having a first reagent flow rate;
   a first mixing chamber in fluid communication with the first reagent channel;
   a first reaction channel in fluid communication with the first mixing chamber;
   a first reaction detection window in fluid communication with the first reaction channel;
   a first reagent outlet well in fluid communication with the first reaction detection window via a first outlet channel; and
   at least one air release valve;
(b) a second reagent flow path comprising:
   a second reagent inlet well;
   a second reagent channel having a depth, a width and a length, in fluid communication with the second reagent inlet well, the second reagent channel having a second reagent flow rate;
   a second mixing chamber in fluid communication with the second reagent channel;
   a second reaction channel in fluid communication with the second mixing chamber;
   a second reaction detection window in fluid communication with the second reaction channel;
   a second reagent outlet well in fluid communication with the second reaction detection window via a second outlet channel; and
   at least one air release valve;
(c) a sample inlet well;
(d) a first sample flow path comprising:
   a first sample channel having a depth, a width and a length, in fluid communication with the sample inlet well and the first mixing chamber of the first reagent flow path, the first sample flow path having a first sample flow path flow rate;
(e) a second sample flow path comprising:
   a second sample channel having a depth, a width and a length, in fluid communication with the sample inlet well and the second mixing chamber of the second reagent flow path, the second sample flow path having a second sample flow path flow rate; and
(f) an oil confiner having a depth, width, and length encompassing the first reagent inlet well, the second reagent inlet well, and the sample inlet well to form a trough for containing an oil in fluid communication with each of the first reagent inlet well, the second reagent inlet well, and the sample inlet well.

These elements are described in more detail below.

Flow Paths from the Inlets to the Mixing Chambers

In an exemplary embodiment, the uACR-Chip can include a first reagent flow path that provides a fluid pathway for combining a first reagent with a biological sample, allowing the first reagent and the biological sample to react to form a first reaction product, and detecting a signal from the first reaction product. The uACR-Chip also can include a second reagent flow path that provides a separate fluid pathway for combining a second reagent with a biological sample, allowing the second reagent and the biological sample to react to form a second reaction product, and detecting a signal from the second reaction product. While the description below focuses on two different reagent pathways, more than two reagent pathways can be included, such as 3, 4, 5, 6, or more reagent pathways.

As shown in FIG. 1, a first reagent channel 110 is in fluid communication with first reagent inlet well 100, a second reagent channel 210 is in fluid communication with second reagent inlet well 200, and a first sample channel 310 and a second sample channel 320 each separately is in fluid communication with sample inlet well 300.

The width of the reagent channels and the sample channels can be the same or different. The width of the reagent channels and the sample channels can be from about 50 to 150 μm. In some configurations, the width of the reagent channels and the sample channels are the same, and are in a range of 75 to 125 μm. In some configurations, the width of the reagent channels and the sample channels are the same, and are about 100 μm.

The depth of the reagent channels and the sample channels can be the same or different. The depth of the reagent channels and the sample channels can be from about 50 to 150 μm. In some configurations, the depth of the reagent channels and the sample channels are the same, and are in a range of 75 to 125 μm. In some configurations, the depth of the reagent channels and the sample channels are the same, and are about 100 μm.

The length of each channel can be the same or can be different. Typically the length of the first reagent channel and the first sample channel is between 5 mm and 50 mm. If the reagent and the sample are to be mixed in equal amounts (at a ratio or about 1:1), the lengths of first reagent channel and the sample channel can be adjusted to produce similar flow rates between the two, and the lengths of the channels can be the same. As will be appreciated by one of skill in the art, in addition to depending on channel dimensions, the flow rate of a given reagent solution can also depend on the fluidic viscosity of the reagent and the biological sample, which one of skill in the art will understand needs to be taken into account when determining flow rate.

In some embodiments, unequal amounts of reagent and biological sample are to be mixed. By adjusting the flow rates, such as by design of the width and the depth of the flow channels, the length of the flow channels, and if necessary including a flow control agent such as a surfactant to reduce surface tension, the flow rates from the reagent inlet and the sample inlet to the mixing chamber can be adjusted to produce a mixing ratio greater than 1:1, such as from about 1.1:1 to 10:1. The flow rates can be adjusted to achieve a mixing ratio of about 1:1, 1.1:1. 1.25:1, 1:1.5, 1:2, 1:2.5, 1:3, 1:3.5, 1:4, 1:4.5, 1:5, 1:5.5, 1:6, 1:6.5, 1:7, 1:7.5, 1:8. 1:8.5, 1:9, 1:9.5, or 1:10. In some embodiments, the amount of biological sample is in excess. For example, a mixing ratio of a biological sample to a detection dye can be from about 1.1:1 to 10:1. In some embodiments, the amount of detection dye is in excess. For example, a mixing ratio of a detection dye to a biological sample can be from about 1.1:1 to 10:1.

For example, if the amount of reagent, such as a dye, is to be mixed with a biological sample at a ratio of 5:1, the width and the depth of the reagent channel and the sample channel can be the same, but the length of the urine sample channel can be 5 times the length of the reagent channel length. In a configuration where the dye channel is 5 mm, the length of urine sample channel would be 25 mm to achieve the desired mixing ratio.

Because of such a design of the uACR-Chip provided herein, accurate measurement of the amount of material added in the inlet wells is not required. Instead, the flow rates designed into the uACR-Chip determine the accurate mixing of the materials as they flow through the device. Thus, in use, a single drop of a fluorescent dye, e.g., for detection of albumin, can be applied to the first reagent inlet, and a single drop of urine can be applied to sample inlet without requiring that a precisely measured or metered amount be applied at either inlet. The design of the uACR-Chip instead regulates the flow of the dye and the urine sample and the two will mix, for example at a 5:1 ratio, in the mixing chamber because of the difference in the flow rates between the two reagent channels, or in some embodiments, because of the difference in the lengths of the two reagent channels.

Similarly, a single drop of a dye, e.g., for detection of creatinine, can be applied to the second reagent inlet without requiring that a precisely measured or metered amount be applied at the inlet. The same single drop of urine already applied to sample inlet can be used for the analysis. The design of the uACR-Chip regulates the flow of the dye and the urine sample and the two will mix at the targeted ratio in the mixing chamber.

The first reagent channel, the second reagent channel, and the first and second sample channels include curved turns to promote or maintain laminar flow through the channels from the inlet wells to the mixing chambers. The degree of bend can be selected to minimize flow restriction through the channels. The smooth channel turnings facilitate mixing ratio control and help to stabilize fluid flow from the inlets to the respective mixing chambers.

A first sample channel 310 is provided from the sample inlet well 300 to the first mixing chamber 120, and a separate second sample channel 320 is provided from the sample inlet well 300 to the second mixing chamber 220. Use of common flow path from the sample inlet as described in conventional microfluidic chips significantly complicates fluidic resistance and therefore flow rate distribution and mixing ratio control. By providing separate flow paths from the sample inlet to each of the mixing chambers, fluidic resistance can be simplified, and better mixing ratio control and flow rate distribution can be achieved.

As will be appreciated by one of skill in the art, the ratio of urine to dye used to detect creatinine dye is dependent on the dye used. Similarly, the ratio of urine to dye used to detect albumin is dependent on the dye used. While each dye will have its optimum mixing ratio with the sample, determination of this mixing ratio is routine experimentation. Once this ratio is known, the lengths of the respective channels can be adjusted accordingly. As can be seen, the chip design is flexible to adjustments to accommodate various targeted mixing ratios and flow rates.

### Mixing Chambers

In the microfluidic devices provided herein, a first mixing chamber is provided to mix a first reagent with a biological sample, such as urine. A second mixing chamber is provided to mix a second reagent with a biological sample, such as urine. Referring to FIG. 1, a first mixing chamber 120 joins the first reagent channel 110 and the first sample channel 310. A second mixing chamber 220 joins the second reagent channel 210 and second sample channel 320.

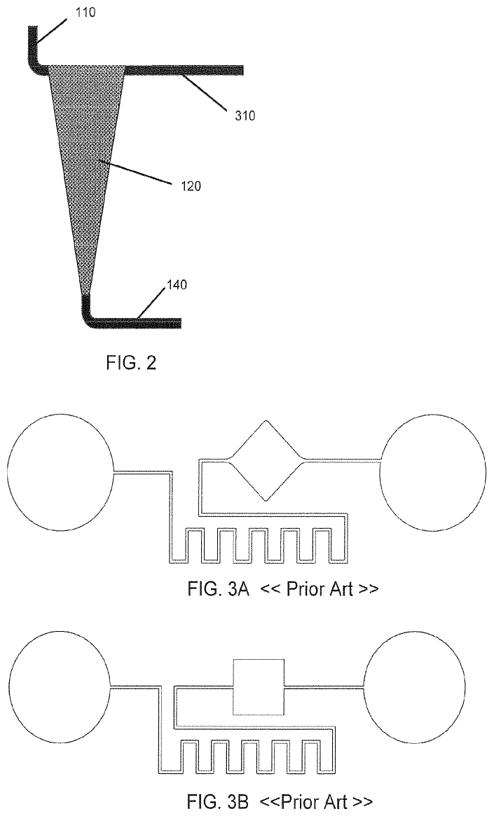

An enlarged view of an exemplary first mixing chamber is shown in FIG. 2. In the configuration shown, the first reagent channel 110 and the first sample channel 310 enter first mixing chamber 120 at opposite positions so that the fluid stream from the first reagent channel 110 and the fluid stream from first sample channel 310 converge directly, causing turbulent flow of the two fluid streams, promoting mixing of the fluids. The mixing chamber has a tapered design, so that a width at the point of entry of the reagent fluid and biological sample fluid is larger than the width at which the mixed product exits the mixing chamber.

The mixing chamber can include baffles or protrusions to direct the flow of mixing fluid streams through the mixing chamber to promote turbulent flow and mixing. When present, the baffles or protrusions can be positioned at any angle relative to the wall of the mixing chamber. The baffles or protrusions can be positioned at any location along a wall of the mixing chamber. In some configurations, one or more baffles can be positioned on each wall of the mixing chamber to form a circuitous route through which the mixing fluid flows must traverse in order to exit the mixing chamber, thereby increasing turbulent flow and mixing of the two fluid streams within the mixing chamber. An angle for the baffles can be under 10 degrees so as not to obstruct any air bubbles that may have been introduced at the initial stage when the uACR-Chip is being filled with liquid. The exit of the mixing chamber is connected to and in fluid communication with a reaction channel. There is no preferred width dimensions at the top (entry) or bottom (exit) of the mixing chamber, as long as the width at the bottom of the chambers is less than the width at the top of the chamber, which assures mixing of the two fluid streams as the two fluid streams merge and flow through the mixing chamber. In some embodiments, the ratio of the width at the top (entry) of the mixing chamber to the width at the bottom (exit) of the mixing chamber is in a range of 2:1 to 5:1. In some embodiments, the width at the top (entry) of the mixing chamber to the width at the bottom (exit) of the mixing chamber is 2:1, or 2.5:1, or 3:1, or 3.5:1, or 4:1, or 4.5:1, or 5:1. In some embodiments, the width at the top (entry) of the mixing chamber is in a range of about 200 to 500 μm and the width at the bottom (exit) of the mixing chamber is in a range of 100 to 150 μm. In some embodiments, the width at the top (entry) of the mixing chamber is about 300 μm and the width at the bottom (exit) of the mixing chamber is about 100 μm.

### Reaction Channels

Reaction channels connect the mixing chamber to a detection window. The length of each of the reaction channels can be separately selected to provide a targeted reaction time the reagent is in contact with the biological sample. The mixing channel length can be selected to be sufficient for complete mixing between the specific reagent and sample, and to provide the desired reaction time after complete mixing. The length of each reaction channel can be from about 5 mm to 150 mm. In some configurations, the length of reach reaction zone separately can be selected to be from about 10 mm to 100 mm.

To configure the microfluidic device to have a relatively small overall dimension, each reaction channel can be designed to include numerous twists and turns or to have a have a “zig zag” configuration. To promote more continuous mixing through the traverse of the reaction channel, the turns can be configured to be at right angles so that fluid flow impacts on a turning wall, promoting turbulent flow and mixing. The fluid dynamics at such impact zones also can promote elimination of dead zones and promotes continuous flow through the fluid circuit.

The width of each of the reaction channels can be the same or different. The width of the reaction channels can be from about 50 to 500 μm. In some configurations, the width of each of the reaction channels are the same, and are in a range of 75 to 150 μm. In some configurations, the width of each reaction channel is wider than the width of the reagent channel and the sample channel connected to the mixing chamber. In some configurations, the width of each of the reaction channels are the same, and are about 100 μm. In some configurations, one or both walls of one or each of the reaction channels and include projections that extend from a wall of the reaction channel into the fluid flowing through the reaction channel to promote turbulent flow of the fluid and increased mixing. In some embodiments, passive mixing microstructures can be integrated into the reaction channel to achieve a thorough and rapid mixing in a reaction channel having a shorter length.

The depth of each of the reaction channels can be the same or different. The depth of each of the reaction channels can be from about 50 to 500 μm. In some configurations, the depth of each of the reaction channels are the same, and are in a range of 75 to 150 μm. In some configurations, the depth of each of the action channels are the same, and are about 100 μm.

The reaction time control for the reaction between the detection dye and creatinine in the sample can be more critical than the reaction between the detection dye and albumin in the sample. Accordingly, the length of the reaction channel for the mixture of creatinine detection dye and biological sample can be longer than the length of the reaction channel for the mixture of the albumin detection dye and biological sample.

### Detection Windows

Each reaction channel is separately connected to a separate detection window. Referring to FIG. 1, first reaction channel 140 is connected to and in fluid communication with first detection window 170, and second reaction channel 240 is connected to and in fluid communication with second detection window 270. The width at the widest point of the detection window can be much greater than the width of the reaction channel connected to the detection window. For example, the width at the widest point of the detection window can be from about 150 μm to 3000 μm. In some embodiments, the width at the widest point of the detection window can be from about 150 μm to 1500 μm, or from about 500 μm to 2500 μm, or from about 1000 μm to 3000 μm.

The length of the detection window, as measured from the entrance to the exit, can be from about 150 μm to 6000 μm. In some embodiments, the length of the detection window, as measured from the entrance to the exit, can be from about 150 μm to 1500 μm, or from about 200 μm to 2500 μm, or from about 300 μm to 3500 μm, or from about 400 μm to 4500 μm, or from about 500 μm to 5500 μm, or from about 600 μm to 6000 μm, or from about 1500 μm to 6000 μm, or from about 2000 μm to 6000 μm.

Through experimentation, it has been determined that rectangular shaped detection windows such as are conventionally used in microfluidic devices, such as shown in FIGS. 3A and 3B, can negatively impact fluidic filling efficiency and flow speed profiles, and can cause air bubble trapping in the detection window. It has been determined that detection windows that contain sharp angles, such as squares, rectangles, and rhomboids do not exhibit the desired fluidic filling efficiency and flow speed profiles.

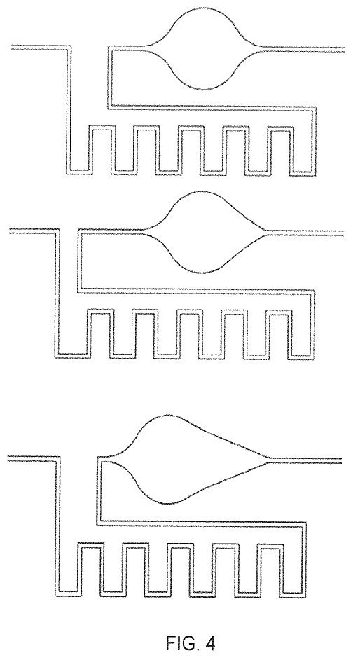

It is preferred, based on experimental testing, computational modeling, and simulation, that the detection window contains no sharp angles, and instead has a smooth curved configuration, which helps to direct fluid flow through the detection window. Exemplary shapes of the detection window include elliptic, biconvex, lenticular, fusiform, ovate, lanceolate, oblanceolate, and tear-drop. These shapes have been found to promote fluidic flow efficiency and create uniform speed profile while avoiding air bubble formation and/or trapping. This can promote enhanced signal detection and quantification based on the detectable signal in the detection window. Exemplary detection window designs are shown in FIG. 4. The final selection of the detection zone shape is based on a comprehensive consideration of fluidic filling efficiency, size, and velocity profile.

To promote uniform filling and fluid flow through the detection window, the detection window can include a flow diverter. Referring to FIG. 1, an exemplary embodiment of the uACR-Chip includes a first window flow diverter 160 in first detection window 170, and a second window flow diverter 260 in first second detection window 270. The flow diverter is positioned at the opening of the detection window where the fluid flowing from the reaction channel enters the detection window. The flow diverter can bifurcate or otherwise modulate the fluid flowing into the detection window to promote fluid completely filling the detection window before exiting the detection window. A plurality of flow diverters can be included in the detection window.

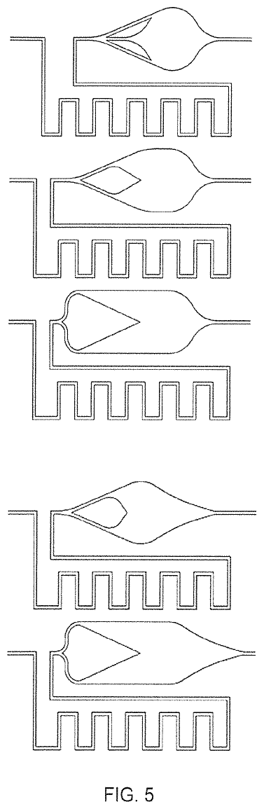

The shape of the flow diverter typically has a smooth curved configuration directing fluid flow around the flow diverter and into the detection window. Exemplary shapes of the flow diverter to be included in the detection window include elliptic biconvex, fusiform, ovate, lanceolate, oblanceolate, and tear-drop. These shapes have been found to promote fluidic flow efficiency and create a uniform speed profile of fluid flowing into the detection window while avoiding air bubble formation. Exemplary detection window designs containing a flow diverter are shown in FIG. 5.

The size and the shape of the flow diverter can be selected to direct the flowing fluid towards the widest part of the detection window and along the fluid flow. Thus, the shape and size of the flow diverter can be predicated on the shape and size of the detection window. In some embodiments, a flow diverter can be selected that has at least a portion of its length that has a similar contour as the detection window. Typically, the length of the flow diverter, which is measured from the end closest to the detection window entrance to the end closest to the detection window exit (corresponding to horizontal as viewed in FIG. 5) can be in the range of from about 300 μm to 3,000 μm, or about 500 μm to 2,500 μm, or about 1,000 μm to 2,000 μm. The width at the widest point of the flow diverter (corresponding to vertical as viewed in FIG. 5) can be in the range of from about 200 μm to 2,950 μm, or about 500 μm to 2,500 μm, or about 750 μm to 2,000 μm.

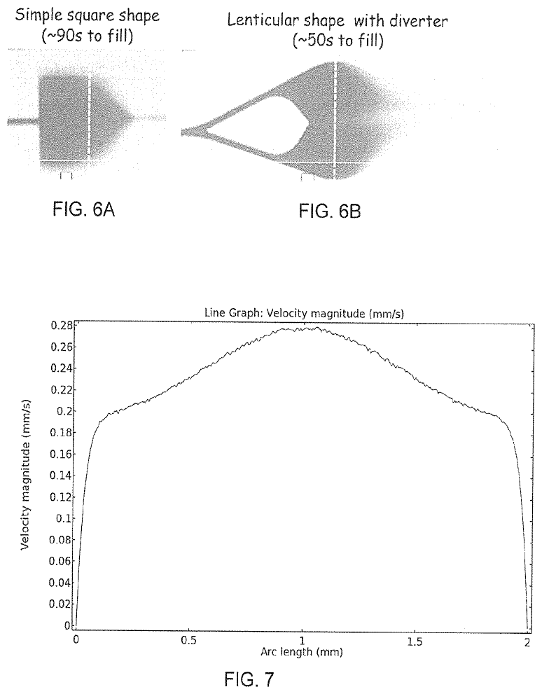

FIGS. 6A and 6B show a comparison of the filling efficiency of a conventional simple square shape detection window compared to a lenticular shape detection window containing a flow diverter as described herein. In the simple square shape detection window, it took approximately 90 seconds to fill the detection window (FIG. 6A). In contrast, the detection window as described herein, having a lenticular shape and containing a flow diverter, completely filled in about 50 seconds.

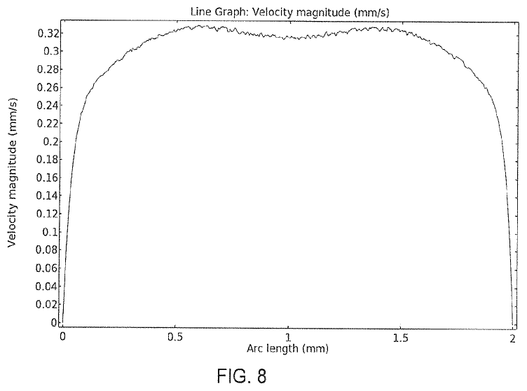

A filling velocity profile of a conventional simple square shape detection window is shown in FIG. 7, and a filling velocity profile of a lenticular shape detection window containing a flow diverter as described herein is shown in FIG. 8. As can be seen from a comparison of the two graphs, the lenticular shape detection window containing a flow diverter exhibits a faster fill velocity.

When flow enters the sharp interface of a much wider square shape detection zone from a narrower mixing/reaction channel (also note the channel entrance to the middle of the detection zone left border in FIG. 7), the flow speed is much faster along the middle region of the square-shaped detection zone compared to the regions closer to the edges. Similar issue at the sharp interface between the flow exit end of the square shape detection zone and the post-detection zone channel occur. This causes nonuniform flow speed in different regions of the detection zone and thus lower filling efficiency. The plots show the flow speed profiles across the detection zone width (the vertical dashed line in the images, which is perpendicular to the horizontal flow direction). Generation of a plateau of flow speed for as large as possible along a portion of the detection zone width is desired. However, in the square shape detection zone, it shows a “hill” shape biphasic speed profile with a sharp peak in the middle as expected. The additional issue of this nonuniform flow speed profile is that the solution in some regions of the detection zone will remain in the detection zone for a longer time before it flows out of the detection zone, which can lead to different reaction time and therefore nonuniform detection signal inside the detection window. In some cases, the significant flow speed nonuniformity can also trap air bubbles in the lower speed region of the detection window that can be hard to remove from the detection window. In contrast, using a smooth shaped detection window as described herein, such as a lenticular shape detection zone, flow speed nonuniformity can be reduced by designing the gradual increasing detection zone width at the flow entrance side and gradual decreasing detection zone width at the flow exit side.

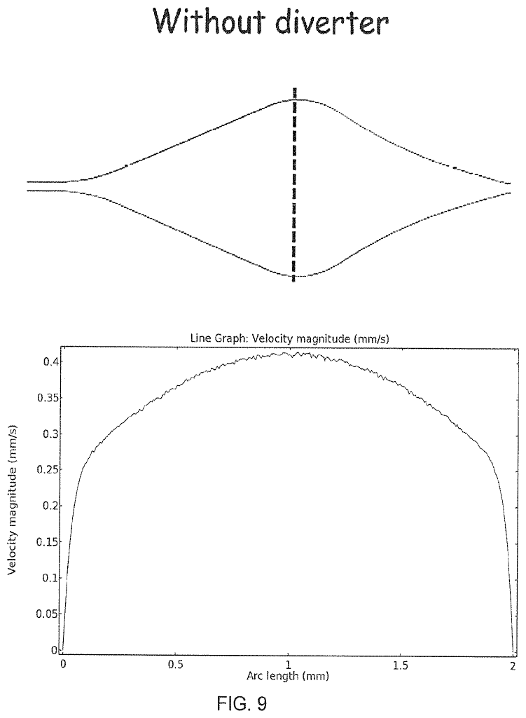

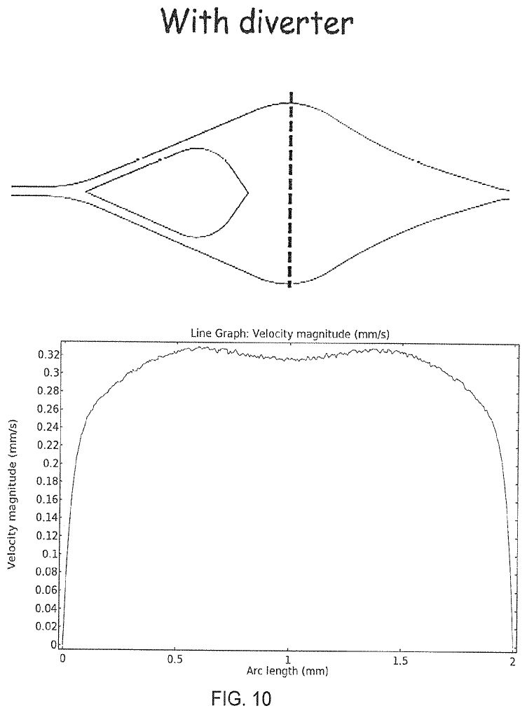

The effect of including a flow diverter is demonstrated in FIGS. 9 and 10. FIG. 9 shows a schematic drawing of a detection window that does not include a flow diverter, and a filling velocity profile is shown. FIG. 10 shows a schematic drawing of a detection window that includes a flow diverter, and a filling velocity profile is shown. A comparison of the filling velocity profiles demonstrates that the presence of the flow diverter increases the speed at which the detection window becomes completely filled with the fluid flowing through the detection window, and allows for a faster measurement of the reaction product flowing through the device. The flow diverter directs the entrance flow along the edges of the detection zone. Collectively, this smooth detection window shape, such as a lenticular shape, combined with the flow diverter in the detection zone successfully achieves the desired uniform flow speed profile (i.e., wider plateau for a large portion of the detection window width).

### Outlet Channels

The reaction fluid flowing through the detection window exits each detection window via an outlet channel connected to, and in fluid communication with, the detection window. The width of the outlet channel is configured to be larger than the width of the reaction channel that is connected to the detection window. The width of the outlet channel is not limited. The larger width of the outlet channel can facilitate air bubble removal from the device. In some configurations, the outlet channel can have a width that is about 110% to 500% of the width of the reaction channel connected to the detection window. For example, if the reaction channel connected to the detection window has a width of 100 μm, the width of the outlet channel can be from about 110 μm to 500 μm

### Outlet Wells

Each outlet channel is connected to its own outlet well. Referring to FIG. 1, an exemplary embodiment of the uACR-Chip includes a first reagent outlet well 190 connected to, and in fluid communication with, first detection window 170 via first outlet channel 180. The uACR-Chip also includes a second reagent outlet well 290 connected to, and in fluid communication with, second detection window 270 via second outlet channel 280.

Because separate outlet wells are provided for reach reaction flow path, fluidic transport along the reaction flow paths can separately be controlled. The separate outlet wells greatly simplifies fluidic transport control for the albumin detection side and the creatinine detection side of the uACR-Chip provided herein.

### Air Release Valves

In the uACR-Chips provided herein, one or more air release valves can be included at various points along the fluid pathways in order to solve any bubble trapping issues.

The air release valves provide an air venting mechanism to allow any entrained air within the pathway to be vented, thereby removing any bubbles that could form and interfere with fluid flow through the channels. The air release valves can be covered with a hydrophobic membrane that allows air migration but prevents reaction liquid from escaping.

In some configurations, an air release valve can be included in the reaction flow path in proximity to the mixing chamber, or in proximity to the detection window, or a first air release valve can be included in proximity to the mixing chamber, and a second air release valve can be included in proximity to the detection window. Referring to FIG. 1, an exemplary embodiment of the uACR-Chip includes an air release valve 130 in first reaction channel 140 in close proximity to the first mixing chamber 120, an air release valve 150 in first reaction channel 140 in close proximity to the first detection window 170, an air release valve 230 in second reaction channel 240 in close proximity to the second mixing chamber 220, and an air release valve 250 in second reaction channel 240 in close proximity to the second detection window 270. In some embodiments, an air release value can be located at a position that is about 1,000 to 2,500 μm from the mixing chamber. The air release valve can be positioned in the first reagent channel 110, or the first sample channel 310, or first reaction channel 140, or any combination thereof. In some embodiments, an air release value can be located at a position that is about 1,000 to 5,000 μm from the entrance to the observation window.

### Oil Confiner

The uACR-Chips provided herein include an oil confiner. An exemplary embodiment is shown in FIG. 7. The oil confiner can form a trough that encompasses all of the inlet wells of the chip, and ensures consistent coverage of the inlet wells with oil contained in the oil confiner. Because of the consistent coverage of all of the inlet wells by the oil, accurate control of flow rate through the various fluid paths can be achieved. The oil confiner confines the oil applied to the chip to cover the inlet wells, and thus minimizes overflow of the oil. Oil overflow of the oil onto the surface of the uACR-Chip could negatively impact optical detection and measurement of the detection signal. Accordingly, the oil confiner can minimize or eliminate any negative impact that oil added to the inlet wells could have on optical detection and measurement of the detection signal.

The oil selected for including in the oil confiner typically has a density that is less than the density of the first reagent, the second reagent, and the biological sample. The density is selected so that the oil floats on the surface of the first reagent, the second reagent, and the biological sample. Oils having a density less than 1 g/mL at 25° C. can be utilized. For example, a silicone oil having a density of 0.98 g/mL or less, mineral oil (density of 0.85 g/ml), a-tocopherol (density of 0.95 g/ml), olive oil (density of 0.92 g/ml), canola oil (density of 0.95 g/ml), octamethyl-cyclotetrasiloxane (density of 0.96 g/ml), or combinations thereof, can be used. Other suitable oils will be readily apparent to one of skill in the art. The oil is selected so that it is immiscible with the first reagent, the second reagent, and the biological sample.

In the uACR-Chip provided herein, a pressure-balancing strategy based on the use of the immiscible oil coverage of the inlet wells can achieve precise control of the mixing ratio of sample and each dye. The uACR-Chip provided herein is based on continuous flow of fluid through the flow channels during signal detection, and the consumption of reagent is very small at any given time during the assay due to the low flow rate in the microfluidic device. In some configurations, 10 μL aliquots of sample and reagents have been found to be sufficient to maintain signal stability at the detection windows for more than 1 hour.

The oil covering the inlet wells and contained within the oil confiner can stabilize and balance the pressure. Pressure differences before adding the oil to the oil confiner are significantly reduced, suggesting that the pressures are balanced. The oil-based pressure balancing strategy can significantly decrease the variation of mixing ratios between different reaction channels, thus improving the detection accuracy of the assay of each reaction channel.

### Method of Fabrication

The uACR-Chips provided herein can be prepared by forming a device pattern and then using the device pattern to produce replica devices.

The device pattern can be designed using any computer design software, such as AUTOCAD (Autodesk, San Rafael, CA) and printed on a transparent film with high resolution. A SU-8 device master can be fabricated on a 3-inch silicon wafer using a standard photolithography process. Standard soft-lithography techniques can be used to make polydimethylsiloxane (PDMS) replica devices from the SU-8 master mold. 3 mm diameter holes can be punched in the PDMS slab as the inlet and outlet reservoirs. Holes also can be punched into the PDMS to form the air release valves. The PDMS slab can be bonded such as by using plasma bonding to a glass slide to seal the channel and yield the uACR-Chip.

The uACR-Chip is filled with water after plasma bonding to preserve hydrophilicity of the channel before use. Additional PDMS is cured around the inlet wells to form a trough to serve as the oil confiner.

When the uACR-Chip is to be used in an analysis of a sample, the inlet and outlet wells are emptied, then sample and reagents are added to their respective wells. Typically, the amount of fluid added to the inlet wells can be 10-100 μL. Once the fluids are added to in the inlet wells, an oil, such as a silicone oil or mineral oil, is added to the oil confiner to cover all of the inlet wells for pressure balancing

### Reagents

Any reagent that can react with albumin to produce a detectable signal can be used for albumin detection. Exemplary reagents include, e.g., Albumin Blue 580 Potassium salt, Square-655 and Square-680-Carboxy dyes (SETA BioMedicals, Urbana, IL), Nile Red (CAS No. 7385-67-3), 8-anilino-1-naphthalenesulfonic acid (ANS), FITC-dextran, rhodamine, rhodamine B isothiocyanate (CAS No. 36877-69-7), bromocresol green (BCG, CAS No. 76-60-8), bromocresol purple (BCP, CAS No. 115-40-2), and Texas red (CAS No. 82354-19-6). For example, the dye reagent from the Albumin Blue Fluorescent Assay Kit™ (Active Motif, 15002) can be used as a reagent and added to the reagent inlet to measure albumin levels in a urine sample.

Any reagent that can react with creatinine to produce a detectable signal can be used for creatinine detection. Picric acid (CAS No. 88-89-1) is a colorimetric dye used in the famous Jaffe reaction for creatinine detection. There are other reagents such as metal nanoclusters, whose fluorescent signal will be quenched after reacting with creatinine. A combination of Rhodamine B dye-gold ion conjugates also can be used.

Other suitable reagents for use as part of a detectable reaction will be readily apparent to one of skill in the art. For example, any suitable reagent used in a commercially available kit for detection of a substrate of interest that produces a detectable reaction can be used as a reagent in the uACR-Chip provided herein. The conditions under which these reactions can be detected are also well-known in the art and can be used with the uACR-Chip provided herein and the methods provided herein.

### Detection

The detectable signal can be, e.g., a fluorescent signal, a colorimetric signal, an electrochemical signal, or a combination thereof. The detectable signal can become stable after about 3-6 minutes after sample/reagent loading, and can last for a long time, e.g., longer than 1 hour.

In some methods, the albumin is detected by use of Albumin Blue 580 Potassium salt (CAS No. 192140-46-8). The Albumin Blue 580 Potassium salt is commercially available (MilliporeSigma, St. Louis, MO; albumin blue kit, Active Motif, Inc., Carlsbad, CA (www.activemotif.com/catalog/104/albumin-flue-fluorescent-assay-kit)). The dye produces a fluorescent signal when combined with albumin. The detectable fluorescent signal becomes stable after a few minutes (e.g., 4-6 minutes) after sample/reagent loading and can last for a long time (e.g., at least 10 minutes and can last for more than 1 hour). The fluorescence signal of albumin can be detected and measured using any device known in the art. For example, the fluorescent signal can be detected using a microscope. In some methods, a Nikon inverted microscope, with a 10× objective and Texas Red filter set can be used for signal reading. A laser diode can be used to provide high power excitation light for fluorescent signal detection in a portable reader for point-of-care applications. A CCD detector can be used to measure the fluorescent signal.

In some methods, the creatinine is detected by use of a picric acid reagent as a detectable dye in a standard Jaffe's reaction. In the presence of creatinine, a quantitive colorimetric reaction occurs. The picric acid reagent is commercially available (e.g., in the Jaffe kit (CR510) from Randox Laboratories-US, Ltd., Kearneysville, WV). The signal becomes stable after about 5-7 minutes after sample/reagent loading and can last for a long time (e.g., at least 10 minutes, and can last for an hour or more). Absorbance of the results of the creatinine reaction can be detected and measured using any device known in the art. For example, a spectrophotometer or a USB microscope can be used.

In some methods, a detection device can, with respect to the detectable signal, detect, read, analyze, quantitate or any combination of these. For example, the detectable signal can be read by a microscope. The microscope can be a digital microscope. The microscope can be a handheld or portable microscope. The microscope can be a USB microscope. The microscope can be a fluorescent microscope. In some configurations, a portable imaging system can be used. A portable imaging system could simplify detection and can be used in a POC test device. A portable imaging system could include photodiode detector. Advances in camera technologies, such as those now incorporated into many smartphones, also could be configured for use in a portable imaging system for detection of the detectable signal. Any optical readers known in the art can be adapted for use to detect the detectable signal.

Any imaging device known in the art can be used for image capture. For example, a portable electronic USB microscope (Dino-Lite USB microscope, Cat. #AD4113T, available from Dunwell Tech, Inc., Torrance, CA, USA) can be used to detect the detectable signal. A microfluidic device can be placed on a stage under the Dino-lite USB Microscope, and different magnifications (e.g., 20×, 30×, or 40×) of the USB microscope can be viewed and the magnification providing the optimum focus of each of the detection windows separately. Appropriate software for image capture and image analysis can be used. Exemplary software for image capture is DinoCapture 2.0 (Version 1.5.41) software. The DinoCapture 2.0 (Version 1.5.41) software with the default settings can be used for acquiring the images, and the “Snapshot” function of the software can be used for recording the color signal that displayed in the detection window.

The color signal then can be analyzed using any image analysis software known in the art to evaluate the captured images. Exemplary image analysis software that can be used to analyze the color images captured by the USB microscope is ImageJ software, an image processing program developed at the National Institutes of Health and the Laboratory for Optical and Computational Instrumentation.

A color image with 1280×1024 pixels that covers the detection window can be captured for color signal measurement. One area of interest (AOI) can be selected using the “Oval Selections” function from ImageJ for each detection window of the device. The color of each AOI can be measured and inverted using “RGB Measure” function from ImageJ, and then can be split into multiple RGB channels, from which the “(R+G+B)/3” channel can be used to calculate the signal intensity. The mean value of “(R+G+B)/3” channel can be recorded and deducted by 255 for the color intensity value of each AOI. Then the average of the two AOI values obtained from top and bottom T1 points can be used as the final color intensity value for each concentration.

The final color intensity values from different concentrations of albumin or creatinine standard samples can be used to create a calibration curve using OriginPro software (OriginLab Corporation, Northampton, MA, USA). The “Linear Fit” function of the OriginPro software then can be applied to check the linearity and limit of detection (LOD). There will be a positive correlation between the intensity of the generated signal and the analyte content in the sample, which can be used to quantify the analytes (albumin and creatinine) present in the sample.

### Methods

As discussed herein, in use, a single drop of the fluorescent dye for detection of albumin can be applied to the first reagent inlet well, a single drop of urine can be applied to the sample inlet well, and a single drop of the dye for detecting creatinine can be applied to the second reagent inlet well. The reagents can be applied without prior measurement, as an exact measured or metered amount is not required. The mixing ratios of the individual dyes with a portion of the biological sample are controlled by the difference in the flow rates of the two reagent channels and the sample channel of the device, or in some embodiments, because of the difference in the lengths of the two reagent channels and the sample channel.

In the methods provided herein, a uACR-Chip provided herein is used. The inlet wells and outlet wells are cleared of residual water. A single drop of the fluorescent dye for detection of albumin can be applied to the first reagent inlet well, a single drop of biological sample, such as urine, can be applied to the sample inlet well, and a single drop of the dye for detecting creatinine can be applied to the second reagent inlet well. A quantity of oil, such as silicone oil, is then applied to the oil confiner to completely cover the reagent wells and sample well. The oil has a density slightly lower than a density of the first reagent solution, a density of the second reagent solution, and the biological sample so that the oil floats on top of the surface of the first reagent solution, the second reagent solution, and the biological sample.

The first reagent flows along the first reagent channel at a first reagent flow rate. The biological sample flows along the first sample channel at a first sample flow path flow rate. The first reagent and the biological sample meet and converge in a first mixing chamber, where turbulent flow mixes the two streams together. The mixing of the first reagent and the biological sample produces a detectable reaction mixture. The detectable reaction mixture then exits the first mixing chamber and flows along a first reaction channel and into a first detection window. The detectable signal of the detectable reaction mixture is detected within the first detection window. The detectable reaction mixture then exits the first detection window via a first outlet channel into a first reagent outlet well.

At the same time, the second reagent flows along the second reagent channel at a second reagent flow rate. The biological sample flows along a second sample channel at a second sample flow path flow rate. The second reagent and the biological sample meet and converge in a second mixing chamber, where turbulent flow mixes the two streams together. The mixing of the second reagent and the biological sample produces a detectable reaction mixture. The detectable reaction mixture then exits the second mixing chamber and flows along a second reaction channel and into a second detection window. The detectable signal of the detectable reaction mixture is detected within the second detection window. The detectable reaction mixture then exits the second detection window via a second outlet channel into a second reagent outlet well.

Once the single drop of fluorescent dye for detection of albumin is applied to the first reagent inlet well, the single drop of biological sample, such as urine, is applied to the sample inlet well, and the single drop of the dye for detecting creatinine is applied to the second reagent well, and oil is added to the oil confiner, gravity will drive the solutions to flow toward each outlet well by virtue of each outlet well being empty. Because of the small dimensions of the channels, the flow can last for longer than 1 hour even though the volume added at the inlets is small. This provides continuous and stable mixing and a stable detectable signal that can be read and measured for comparatively long periods of time. The flow is continuous until the pressure difference between the inlets and outlets is balanced.

The reagents do not need to be added simultaneously because the signal doesn't decay because of the continuous mixing that occurs within the microfluidic device. While the reagents in the two reagent inlet wells and the biological sample in the sample inlet well do not need to be added simultaneously, the time gap should be smaller than the time required for one reagent to travel from a reagent inlet to the mixing chamber. Depending on the length of the channels, the time available for adding the reagents and biological sample can be a few minutes. This can simplify use of the device by the end user because precise timing of sample and dye application is not required.

The biological sample can be diluted prior to it being applied to the sample inlet well. For example, the biological sample can be diluted to a suitable concentration so as to fall within the detection range. The biological sample can be diluted with water, saline, or a buffered saline.

In the methods provided herein, the quantity of the first reagent, the quantity of the second reagent, and the quantity of the biological sample applied to the microfluid device provided herein can be applied without measurement of the amount being applied to the respective inlets. As will be appreciated by one of skill in the art, this removes a considerable source of variability in reactions. The microfluidic device provided herein and methods of using the device do not require that a precise amount of reagents and biological sample used in the reaction be applied to the inlet wells. In the methods provided herein, each reagent and biological sample can be applied to their respective inlet as a single drop. In some methods, the single drop can be in the range of 15 μL to 75 μL.

In some methods, the dye for producing a detectable signal and the biological sample are to be mixed in equal amounts, or at a ratio of 1:1. In some methods, the dye for producing a detectable signal and the biological sample are to be mixed in unequal amounts, or at a ratio that is not 1:1. In some embodiments of the invention, the first reagent and the biological sample are mixed at unequal amounts. For example, for the detection of albumin with the dye reagent from the Albumin Fluorescent Assay Kit™, the suggested mixing ratio of sample:dye is 1:6.

The user of the microfluidic device provided herein does not need to do anything more than apply the reagents and the sample to the device. The device is designed to take into account the optimum mixing ratio of dye to sample. For example, the microfluidic device includes an engineered difference in the flow rates to produce the desired ratio of sample to dye to produce the detectable signal. The engineered differences in the microfluidic device include channel dimensions, as well as consideration of the fluidic viscosity of a given reagent solution, which are taken into account when determining and designing flow rate and dimensions of each of the channels of the microfluidic device.

For example, in some embodiments, the width and depth of the first reagent channel and the first sample channel are the same but the length of the first sample channel can be 6 times that of the first reagent channel, for example, 36 mm for the first sample channel and 6 mm for the first reagent channel. The first reagent and the first biological sample will mix inside the first mixing chamber at a ratio of sample:dye of 1:6 because of the difference in the lengths of the two reagent channels.

In some embodiments, a difference in the flow rates can be used to provide the desired ratio. In the microfluidic devices provided herein, because the pressures at the two inlets are the same by virtue of the oil in the oil confiner, the mixing ratio can be calculated using the following equation:

$$Mixing ratio = \frac{{Q}_{1}}{{Q}_{2}} = \frac{{R}_{2}}{{R}_{1}} = \frac{{L}_{2}}{{L}_{1}}$$

where Q₁ is the flow rate of channel 1, Q₂ is the flow rate of channel 2; R₁ is the flow resistance of channel 1, R₂ is the flow resistance of channel 2; and L₁ is the length of channel 1, R₂ is the length of channel 2.

Due to the small dimension of the microfluidic channel, the flow inside the channel is considered to be laminar flow. Both the urine and dye solution are water based solutions with very low concentration of solutes, and thus it is reasonable to assume they have approximately the same density and viscosity. In one embodiment of the uACR-Chip provided herein, the reagent channels and biological sample channels have a rectangular shape with the same width and depth. Accordingly, R₁ is proportional to L₁, and R₂ is proportional to L₂. Therefore, the mixing ratio can be determined by using various ratios of L₂ to L₁.

As will be appreciated by one of skill in the art, such a method can be used to mix any reagent with a biological sample that need to be added in unequal amounts. Furthermore, the amount of each reagent and biological sample being added to an inlet well of the microfluidic device provided herein does not need to be accurately measured or measured at all for the proper reaction to take place, that is, for the reaction to take place at the appropriate ratio.

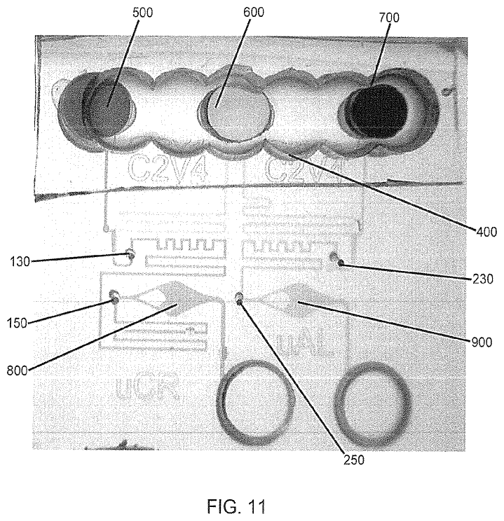

An exemplary embodiment is shown in FIG. 11. FIG. 11 is a photograph showing the detection of creatinine and albumin in a urine sample using a colorimetric dye and fluorescent dye. In the example shown in FIG. 11, the creatinine detection dye 500 is Randox Jaffe Creatinine Reagent (which contains picric acid, sodium hydroxide, and surfactants). The albumin detection dye 700 is albumin blue 580. As can be seen in FIG. 11, in this embodiment, the urine sample 600 to be tested for creatinine and albumin is applied to a central sample inlet well. A creatinine detection dye 500 is applied to a first reagent inlet well (left side in the figure). An albumin detection dye 700 is applied to a second reagent inlet well (right side in the figure). Oil is then added to the oil confiner 400. An exemplary oil is a silicone oil that has a density less than the density of the urine and dye solutions so that the oil floats on the surface of the urine and dye solutions. The oil in the oil confiner 400 equalizes the pressure amongst all of the inlets, and the fluids in the inlets begin to flow.

The creatinine detection dye 500 travels along a first reagent channel and the urine sample 600 travels along a first sample channel. The first reagent channel and the first sample channel are connected to a first mixing chamber, where the flow of creatinine detection dye 500 and urine sample 600 converge and are mixed by turbulent flow within the mixing chamber. The reaction mixture exits the first mixing chamber via a first reaction channel, and passes an air release value that can vent any entrained air to minimize or prevent bubble formation. The reaction mixture traverses the circuitous first reaction channel to provide time for mixing and reaction stabilization, passes another air release value that can vent any entrained air to minimize or prevent bubble formation, and enters a first detection window, where the detectable signal can be detected and measured.

The first detection window has a greater volume than the first reaction channel, for easy detection of the colorimetric reaction between the creatinine detection dye and any creatinine in the urine sample. For example, the width at the widest point of the detection window can be from about 150 μm to 3000 μm, and the length of the detection window, as measured from the entrance to the exit, can be from about 150 μm to 6000 μm.

The albumin detection dye 700 travels along a second reagent channel and the urine sample 600 travels along a second sample channel. The second reagent channel and the second sample channel are connected to a second mixing chamber, where the flow of albumin detection dye 700 and urine sample 600 converge and are mixed by turbulent flow within the mixing chamber. The reaction mixture exits the second mixing chamber via a second reaction channel, and passes an air release value that can vent any entrained air to minimize or prevent bubble formation. The reaction mixture traverses the circuitous second reaction channel to provide time for mixing and reaction stabilization, passes another air release value that can vent any entrained air to minimize or prevent bubble formation, and enters a second detection window, where the detectable signal can be detected and measured. The second detection window has a greater volume than the second reaction channel, for easy detection of the fluorescent reaction between the albumin detection dye and any albumin in the urine sample.

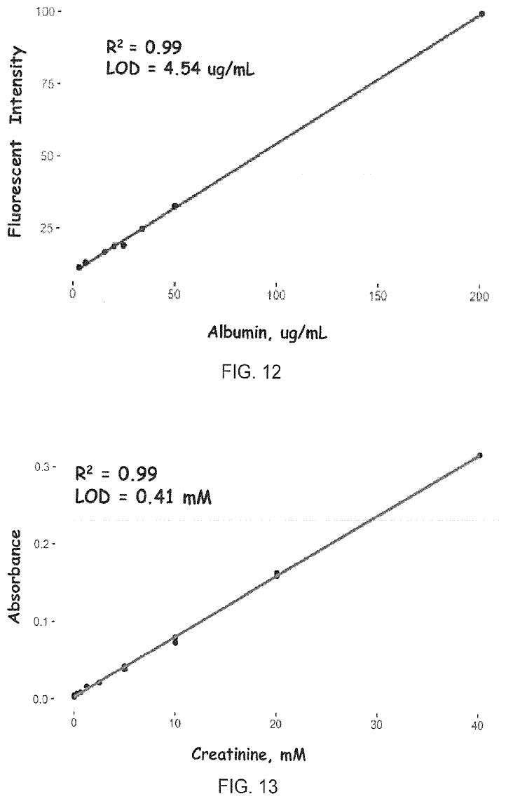

The uACR-Chip has been tested and demonstrated a calibration curve for the albumin detection part of the uACR-Chip to have an R² of 0.99 and a limit of detection (LOD) of 4.64 μg/ml using albumin standards, which is below the 30 μg albumin per ml urine level considered to be indicative of kidney damage. The results are shown in FIG. 12. The uACR-Chip demonstrated a calibration curve for the creatinine detection part of the uACR-Chip to have an R² of 0.99 and a limit of detection (LOD) of 0.41 mM using creatinine standards. The results are shown in FIG. 13.

In comparison to existing assays for measurement of albumin or creatinine in a sample, the uACR-Chip provided herein has the following notable advantages: 1) low cost, 2) easy operation, 3) quantitative and stable measurement, and 4) compatibility for portable and inexpensive optical readers.

As will be appreciated by one of skill in the art, the method of the invention may be used to monitor kidney damage of an individual, as a means of monitoring disease progression. The method of the invention may also be used for screening at-risk individuals, for example, individuals with a familial history of chronic kidney disease, with diabetes mellitus, high blood pressure or glomerulonephritis.

Individuals diagnosed with chronic kidney disease and who show signs of the disease worsening or anyone who is being monitored regularly and shows signs of the disease worsening may be prescribed medication to reduce blood pressure or may be assigned kidney protective treatments such as aggressive BP lowering, Renin Angiotensin Aldosterone System Inhibitors (RAASi), Sodium Glucose Co-transporter-2 inhibitors (SGLT2i), or any combination thereof. The Cystatin C based eGFR (estimated glomerular filtration rate) method makes feasible more frequent and even daily monitoring of kidney status in patients at high risk either of AKI (acute kidney injury), or of acute or chronic deterioration requiring dialysis initiation, a strategy which could help avoid unplanned emergency room visits or emergency dialysis starts.

### EXAMPLES

The invention will now be further explained by way of examples. The following examples are included for illustrative purposes only and are not intended to limit the scope of the embodiments provided herein.

### Example 1. Fabrication of the uACR-Chips

An exemplary uACR-Chip was prepared by forming a device pattern and then using the device pattern to produce replica devices. The device pattern was that shown in FIG. 1.

The device pattern was designed using AUTOCAD (Autodesk, San Rafael, CA) computer design software, and printed on a transparent film with high resolution. Standard SU-8 based photolithography was used to make a device master mold, which was fabricated on a 3-inch silicon wafer. Standard soft-lithography techniques were used to make polydimethylsiloxane (PDMS) replica devices from the SU-8 master mold. Holes were punched in the PDMS slab as the inlet and outlet reservoirs, and to form air release valves. The PDMS slab was bonded by plasma bonding to a glass slide to seal the channel and yield the uACR-Chip. The uACR-Chip was filled with water after plasma bonding to preserve hydrophilicity of the channel before use. Additional PDMS then was cured around the inlet wells to form a trough to serve as the oil confiner.

### Example 2. Albumin Detection Using the uACR-Chip

The dye reagent (Albumin Blue 580 Potassium salt) and standard human serum albumin (HSA) were prepared according to the product datasheets. Raw urine samples or urine samples diluted with Dulbecco's phosphate-buffered saline (DPBS) were used as the test samples. The dye solution and test samples were added to the corresponding inlets in the uACR-Chip microfluidic device using a dropper. Oil was then added to the oil confiner to cover the inlets. The albumin dye and the urine sample entered the mixing chamber, where they were mixed, and the resulting reaction mixture exited the mixing chamber and traversed a reaction channel connected to a detection window, into which the reaction mixture flowed. It took about 1-2 minutes until the reaction mixture entered the detection window. The fluorescent signal was recorded by a Nikon inverted microscope, with a 10× objective and Texas Red filter.

The signals of standard HSA were used to construct a calibration curve. The concentrations of the unknown samples were calculated according to the calibration curve. The LOD is calculated from the regression line of the calibration curve (3*standard error of regression/slope). Each test has two repeats. We also tested the albumin level using the well-plate method as a control. The sample and dye were loaded and mixed in the wells using a pipette at a fixed ratio. The signal was recorded by a multi-plate reader (Synergy 4 HT). Each test sample was assayed in duplicate.

The calibration curve was plotted after subtracting a blank signal. The results are shown in FIG. 12. The R² value of the linear fit is 0.99. The LOD calculated from regression line is 4.54 g/ml, which is >3 times lower than the normal range (clinical cut-off level: about 20 μg/ml).

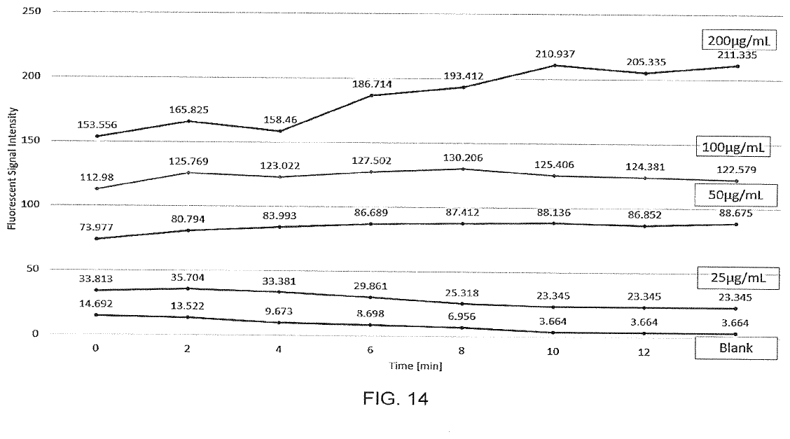

Compared to the traditional well-plate method, the uACR-Chip microfluidic device provided herein also showed great advantage in signal stability due to its continuous mixing property. In the well-plate method, the sample and dye signal is premixed and loaded into a well. The fluorescent signal decays slowly due to concentration changes caused by solvent evaporation. In the uACR-Chip microfluidic device, fresh reagents flow into the detection zone continuously; the sealed channel and the oil that covers the reagent wells prevent evaporation. As illustrated in FIG. 14, the signal of the uACR-Chip was stable for at least 10 minutes, and results indicated stability for up to 1 hour.

FIG. 14 shows the results of monitoring of fluorescent signal intensity of albumin detection on the uACR-Chip microfluidic device over time to show the kinetics and stability. The different lines in the figure are for different albumin concentrations, which varied from 0 μg/mL to 200 μg/mL.

The uACR-Chip demonstrates a linear relationship between concentration and fluorescent intensity in 0-200 μg/ml HSA standards and the LOD is below the normal range of albumin level in urine. In addition, the significant signal stability of the uACR-Chip decreases detection inaccuracy caused by variations in measurement time points. As will be appreciated by one of skill in the art, using the conventional test, the signal can decay by almost 50% within one hour whereas using the method described herein, the signal is stable for over an hour. This signal decay in the prior art methods is significant because a signal that is read too late may miss an individual who has albumin levels of 30-40 μg/ml (which is an indication of kidney disease).

### Example 3. Creatinine Detection Using the uACR-Chip

The creatinine dye reagent (Randox Jaffe Creatinine Reagent (which contains picric acid, sodium hydroxide, and surfactants)) and standard creatinine were prepared according to the product datasheets. Raw urine samples or urine samples diluted with Dulbecco's phosphate-buffered saline (DPBS) were used as the test samples. The dye solution and test samples were added to the corresponding inlets in the uACR-Chip microfluidic device using a dropper. Oil was then added to the oil confiner to cover the inlets. The creatinine dye and the urine sample entered the mixing chamber, where they were mixed, and the resulting reaction mixture exited the mixing chamber and traversed a reaction channel connected to a detection window, into which the reaction mixture flowed. It took about 1-2 minutes until the reaction mixture entered the detection window. The colorimetric signal was recorded using a Thorlab spectrometer for absorbance signal reading at 492 nm. The signals of standard creatinine were used to construct a calibration curve. The concentrations of the unknown samples were calculated according to the calibration curve. The LOD is calculated from the regression line of the calibration curve (3*standard error of regression/slope). Each test has two repeats. We also tested the creatinine level using the well-plate method as a control. The sample and dye were loaded and mixed in the wells using a pipette at a fixed ratio. The signal was recorded by a multi-plate reader (Synergy 4 HT). Each test sample was assayed in duplicate.

The calibration curve was plotted after subtracting a blank signal. The results are shown in FIG. 13. The R² value of the linear fit is 0.99. The LOD calculated from regression line is 0.41 mM. In comparison, the commercial ACR kit with DCA Vantage has a LOD of 1.3 mM.

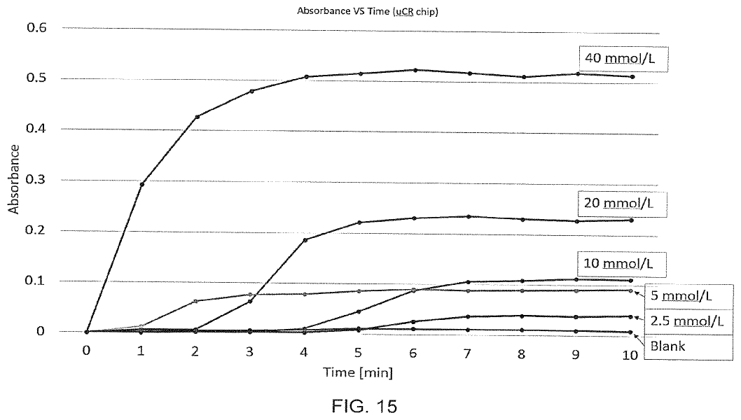

Compared to the traditional well-plate method, the uACR-Chip microfluidic device provided herein also showed great advantage in signal stability due to its continuous mixing property. In the well-plate method, the sample and dye signal is premixed and loaded into a well. The absorbance can change slowly due to concentration changes caused by solvent evaporation. In the uACR-Chip microfluidic device, fresh reagents flow into the detection zone continuously; the sealed channel and the oil that covers the reagent wells prevent evaporation. As illustrated in FIG. 15, the absorbance of the uACR-Chip was stable for at least 10 minutes, and results indicated stability for up to 1 hour.

FIG. 15 shows the results of monitoring of absorbance signal intensity of creatinine detection on the uACR-Chip microfluidic device over time to show the kinetics and stability. The different lines in the figure are for different creatinine concentration, from 0 mM to 40 mM.

### Example 4. Clinical Sample Validation

The uACR-Chip microfluid chip provided herein was further validated using archived patient urine samples from patients who have been diagnosed with CKD.

The albumin and creatinine concentrations in the archived patient urine samples were tested in the respective portions of the uACR-Chip microfluidic device provided herein and compared with a commercial uACR kit and bench top chemistry analyzer (i.e., ACR kit with DCA Vantage analyzer). The resulting measurements used for the comparison analysis are shown in Table 1. The microfluidic chip test for albumin, creatinine and uACR of 10 frozen stored CKD urine samples were quantitatively compared as an Alternate Method to the commercial DCA Vantage ACR kit test. This was done statistically using the Deming regression method and the Passing-Bablok regression method by the EP Evaluator® available at Shared Health Manitoba. The results of the Passing-Bablok regression are shown below in Table 2. Also provided are the regression slope, intercept and correlation coefficient for each biomarker test data that were compared.

TABLE 1
Comparison of uACR-Chip to commercial assays

| | DCA Reading | | | Chip Reading | | |
|---|---|---|---|---|---|---|
| | Albumin | Creatinine | uACR | Albumin | Creatinine | uACR |
| Sample ID | μg/ml | mM | mg/mmol | μg/ml | mM | mg/mmol |
| POC120 | 268.2 | 2.8 | 95.8 | 275.2 | 3.1 | 88.8 |
| POC121 | 7.1 | 9.9 | 0.7 | 13.3 | 11.3 | 1.2 |
| POC122 | <5.0 | 6.3 | <0.8 | 6.7 | 6.1 | 1.1 |
| POC124 | 36.3 | 7.6 | 4.8 | <5.0 | 7.6 | <0.7 |
| POC125 | >300 | 15.5 | >19.4 | >300 | 14.9 | >20.1 |
| POC126 | 24.5 | 7 | 3.5 | 23.7 | 10.8 | 2.2 |
| POC127 | >300.0 | 2.5 | >120.0 | 298.3 | 3.9 | 76.5 |
| POC129 | >300 | 10.8 | >27.8 | >300 | 7.8 | >38.5 |
| POC130 | >300.0 | 8.2 | >36.6 | >300 | 13.5 | >22.2 |
| POC135 | 292.4 | 1.9 | 153.9 | >300 | 4.7 | >63.8 |

TABLE 2
Statistical Analysis Results

| Y | X | | | | | Corr |
|---|---|---|---|---|---|---|
| Method | Method | Analyte | N | Slope | Intercept | Coeff (R) |
| uACR | DCA | U ACR | 10/10 | 0.633 | 0.59 | 0.8910 |
| Chip | Reading | U Albumin | 10/10 | 1.000 | 0.06 | 0.9974 |
| Reading | | U Creatinine | 10/10 | 1.000 | 0.85 | 0.8369 |

The results indicate the data passed the comparison test for albumin, creatinine and uACR.

### LISTING OF FIGURE REFERENCE NUMBERS

100 first reagent inlet well
110 first reagent channel
120 first mixing chamber
130 air release valve
140 first reaction channel
150 air release valve
160 first window flow diverter
170 first detection window
180 first outlet channel
190 first reagent outlet well
200 second reagent inlet well
210 second reagent channel
220 second mixing chamber
230 air release valve
240 second reaction channel
250 air release valve
260 second window flow diverter
270 second detection window
280 second outlet channel
290 second reagent outlet well
300 sample inlet well
310 first sample channel
320 second sample channel
400 oil confiner
500 creatinine detection dye
600 urine sample
700 albumin detection dye
800 creatinine detection window
900 albumin detection window
1000 microfluidic device

1-27. (canceled)

28. A microfluidic device, comprising:
(a) a first reagent flow path comprising:
a first reagent inlet well;
a first reagent channel having a depth, a width and a length, in fluid communication with the first reagent inlet well, the first reagent channel having a first reagent flow rate;
a first mixing chamber in fluid communication with the first reagent channel;
a first reaction channel in fluid communication with the first mixing chamber;
a first reaction detection window in fluid communication with the first reaction channel;
a first reagent outlet well in fluid communication with the first reaction detection window via a first outlet channel; and
at least one air release valve;
(b) a second reagent flow path comprising:
a second reagent inlet well;
a second reagent channel having a depth, a width and a length, in fluid communication with the second reagent inlet well, the second reagent channel having a second reagent flow rate;
a second mixing chamber in fluid communication with the second reagent channel;
a second reaction channel in fluid communication with the second mixing chamber;
a second reaction detection window in fluid communication with the second reaction channel;
a second reagent outlet well in fluid communication with the second reaction detection window via a second outlet channel; and
at least one air release valve;
(c) a sample inlet well;
(d) a first sample flow path comprising:
a first sample channel having a depth, a width and a length, in fluid communication with the sample inlet well and the first mixing chamber of the first reagent flow path, the first sample flow path having a first sample flow path flow rate;
(e) a second sample flow path comprising:
a second sample channel having a depth, a width and a length, in fluid communication with the sample inlet well and the second mixing chamber of the second reagent flow path, the second sample flow path having a second sample flow path flow rate; and
(f) an oil confiner having a depth, width, and length encompassing the first reagent inlet well, the second reagent inlet well, and the sample inlet well to form a trough for containing an oil in fluid communication with each of the first reagent inlet well, the second reagent inlet well, and the sample inlet well.

29. The microfluidic device of claim 28, wherein the first reaction detection window and the second reaction detection window each separately has a shape that is elliptic, biconvex, lenticular, fusiform, ovate, lanceolate, oblanceolate, or tear-drop.

30. The microfluidic device of claim 28, wherein the first reaction detection window and the second reaction detection window each comprises a window flow diverter.

31. The microfluidic device of claim 30, wherein:
the window flow diverter in the first reaction detection window has a shape that is elliptic, biconvex, lenticular, fusiform, ovate, lanceolate, oblanceolate, or tear-drop; and
the window flow diverter in the second reaction detection window has a shape that is elliptic, biconvex, lenticular, fusiform, ovate, lanceolate, oblanceolate, or tear-drop.

32. The microfluidic device of claim 28, wherein:
the first mixing chamber is tapered and has a width at it widest point of 200 to 500 μm a width at its narrowest point of 100 to 150 μm; and
the second mixing chamber is tapered and has a width at it widest point of 200 to 500 μm a width at its narrowest point of 100 to 150 μm.

33. The microfluidic device of claim 28, wherein:
the first reagent channel and the first sample channel are connected to the first mixing chamber at opposite positions so that a fluid stream from the first reagent channel and a fluid stream from first sample channel converge within the first mixing chamber to create turbulence; and
the second reagent channel and the second sample channel are connected to the second mixing chamber at opposite positions so that a fluid stream from the second reagent channel and a fluid stream from second sample channel converge within the second mixing chamber to create turbulence.

34. The microfluidic device of claim 28, wherein:
the first reagent channel and the second reagent channel along the major part of their length have the same cross-sectional shape.

35. The microfluidic device of claim 28, wherein the first reagent channel and the second reagent channel each independently include at least one section having a zig zag shape comprising turns at right angles.

36. The microfluidic device of claim 28, wherein:
a width of the first outlet channel is about 110% to 500% of the width of the first reaction channel; and
a width of the second outlet channel is about 110% to 500% of the width of the second reaction channel.

37. The microfluidic device of claim 28, wherein:
the at least one air release valve of the first reagent flow path is located before and in close proximity to the first reaction detection window; and
the at least one air release valve of the second reagent flow path is located before and in close proximity to the second reaction detection window.

38. The microfluidic device of claim 37, wherein:
the first reagent flow path includes a second air release valve located after and in close proximity to the first mixing chamber; and
the second reagent flow path includes a second air release valve located after and in close proximity to the second mixing chamber.

39. A method of detecting albumin and creatinine in a single drop of biological fluid sample to diagnose or monitor a chronic kidney disease, the method comprising:
providing a microfluidic device of claim 28;
applying a solution of a fluorescent dye for detection of albumin to the first reagent inlet well;
applying a biological sample to the sample inlet well;
applying a solution of a dye for detecting creatinine to the second reagent inlet well;
applying an oil to the oil confiner to completely cover the reagent inlet wells and the sample inlet well;
allowing the solution of a fluorescent dye for detection of albumin and the biological sample to mix in the first mixing chamber to form a first reaction product that flows to the first detection window;
allowing the solution of the dye for detection of creatinine and the biological sample to mix in the second mixing chamber to form a second reaction product that flows to the second detection window;
detecting a detectable signal in the first detection window; and
detecting a detectable signal in the second detection window.

40. The method of claim 39, wherein the oil has a density lower than a density of the solution of a fluorescent dye for detection of albumin, a density of the solution of the dye for detection of creatinine, and the biological sample so that the oil floats on top of a surface of the solution of a fluorescent dye for detection of albumin, the solution of the dye for detection of creatinine, and the biological sample.

41. The method of claim 39, wherein the biological sample is urine.

42. The method of claim 39, wherein the fluorescent dye for detection of albumin comprises albumin blue 580 Potassium salt, Square-655 dye, Square-680-Carboxy dye, Nile Red, 8-anilino-1-naphthalenesulfonic acid (ANS), FITC-dextran, rhodamine, bromocresol green (BCG), bromocresol purple (BCP), Texas Red, or a combination thereof.

43. The method of claim 39, wherein the dye for detection of creatinine comprises picric acid.

44. A kit, comprising:
a microfluidic device claim 28; and
instructions for use thereof.

45. The kit of claim 44, further comprising:
a solution of a fluorescent dye for detection of albumin; and
a solution of a dye for detection of creatinine.

46. The kit of claim 45, the fluorescent dye for detection of albumin comprises albumin blue 580 Potassium salt, Square-655 dye, Square-680-Carboxy dye, Nile Red, 8-anilino-1-naphthalenesulfonic acid (ANS), FITC-dextran, rhodamine, bromocresol green (BCG), bromocresol purple (BCP), Texas Red, or a combination thereof.

47. The kit of claim 45, wherein the dye for detection of creatinine comprises picric acid.
